(19) United States
(12) Patent Application Publication
(10) Pub. No.: US 2024/0062857 A1
(43) Pub. Date: Feb. 22, 2024

(54) SYSTEMS AND METHODS FOR VISUALIZATION OF MEDICAL RECORDS

(71) Applicant: Shanghai United Imaging Intelligence Co., Ltd., Shanghai (CN)

(72) Inventors: Benjamin Planche, Briarwood, NY (US); Ziyan Wu, Lexington, MA (US); Meng Zheng, Cambridge, MA (US)

(73) Assignee: Shanghai United Imaging Intelligence Co., Ltd., Shanghai (CN)

(21) Appl. No.: 17/891,625

(22) Filed: Aug. 19, 2022

(51) Int. Cl. G16H 10/60; G16H 30/20; G06T 17/20

(52) U.S. Cl. CPC G16H 10/60; G16H 30/20; G06T 17/20

(57) ABSTRACT

A two-dimensional (2D) or three-dimensional (3D) representation of a patient may be provided (e.g., as part of a user interface) to enable interactive viewing of the patient's medical records. A user may select one or more areas of the patient representation. In response to the selection, at least one anatomical structure of the patient that corresponds to the selected areas may be identified based on the user selection. Medical records associated with the at least one anatomical structure of the patient may be determined based on one or more machine-learning models trained for detecting textual or graphical information associated with the at least one anatomical structure in the one or more medical records. The one or more medical records may then be presented, e.g., together with the 2D or 3D representation of the patient.

### BACKGROUND

Hospitals, clinics, laboratories and medical offices may create large volumes of patient data (e.g., medical records) during the course of their healthcare activities. For example, laboratories may produce patient data in numerous forms, from x-ray and magnetic resonance images to blood test concentrations and electrocardiograph data. Means for accessing these medical records, however, are limited, and are generally textual (e.g., typing in a patient's name and seeing a list of diagnoses/prescriptions) and/or one-dimensional (e.g., focusing on one specific category at a time). There are no good ways for aggregating and visualizing the medical records of a patient, let alone doing so in an interactive manner.

### SUMMARY

Described herein are systems, methods and instrumentalities associated with accessing and visually interacting with a patient's medical records. The systems, methods and/or instrumentalities may utilize one or more processors configured to generate a two-dimensional (2D) or three-dimensional (3D) representation of a patient (e.g., as part of a graphical user interface (GUI) of a medical records application). The one or more processors may be further configured to receive a selection (e.g., by a user such as the patient or a doctor) of one or more areas of the 2D or 3D patient representation, and identify at least one anatomical structure of the patient that corresponds to the one or more areas of the 2D or 3D representation based on the user selection. Based on the identified anatomical structure(s), the one or more processors may determine one or more medical records associated with the anatomical structure(s), for example, using a first machine-learning (ML) model trained for detecting textual or graphical information associated with the anatomical structure(s) in the one or more medical records. The one or more processors may then present the one or more medical records of the patient, for example, together with the 2D or 3D representation of the patient (e.g., as part of the GUI for the medical records application). For instance, the one or more medical records may be presented by overlaying the 2D or 3D representation of the patient with the one or more medical records and displaying the 2D or 3D representation of the patient overlaid with the one or more medical records. In examples wherein the one or more medical records may include medical scan images of the patient, the scan images may be registered before being displayed with the 2D or 3D representation.

In some embodiments described herein, the 2D or 3D representation described herein may include a 2D or 3D human mesh generated using a second ML model trained for recovering the 2D or 3D human mesh base on one or more pictures of the patient or one or more medical scan images of the patient. In some embodiments described herein, the one or more processors described herein may be configured to modify the 2D or 3D human mesh of the patient based on the one or more medical records determined by the first ML model.

In some embodiments described herein, the one or more medical records may include a medical scan image of the patient and the first ML model may include an image classification and/or segmentation model trained for automatically recognizing that the medical scan image is associated with the at least one anatomical structure. In some embodiments described herein, the one or more medical records may include a diagnosis or prescription for the patient and the first ML model may include a text processing model trained for automatically recognizing that the diagnosis or prescription includes texts associated with the at least one anatomical structure.

In some embodiments described herein, the 2D or 3D representation of the patient may include multiple views of patient and the one or more processors may be further configured to switch from displaying a first view of the patient to displaying a second view of the patient based on a user input. The first view may, for example, depict a body surface of the patient, while the second view may depict one or more anatomical structures of the patient. In some embodiments described herein, the one or more processors may be configured to receive an indication that a medical record among the one or more medical records of the patient has been selected, determine a body area associated with the selected medical record, and indicate the body area associated with the selected medical record on the 2D or 3D representation of the patient.

### BRIEF DESCRIPTION OF THE DRAWINGS

A more detailed understanding of the examples disclosed herein may be had from the following description, given by way of example in conjunction with the accompanying drawings.

FIGS. 1A-1B are simplified diagrams illustrating graphical user interfaces (GUIs) for visually interacting with a patient's medical records, according to some embodiments described herein.

FIGS. 2A-2B are simplified diagrams further illustrating the GUIs for interacting with the patient's medical records, according to some embodiments described herein.

FIG. 3 is a flow diagram illustrating an example method for determining medical records of a patient based on selected areas of a graphical representation of the patient and displaying the records together with the graphical representation, according to some embodiments described herein.

FIG. 4 is a flow diagram illustrating an example method for generating the 3D human mesh to represent the patient based on pictures and/or medical scan image(s) of the patient, according to some embodiments described herein.

FIG. 5 is a flow diagram illustrating an example method for determining areas of the graphical representation of the patient based on selected medical records of the patient and indicating the determined areas on the graphical representation, according to some embodiments described herein.

FIG. 6 is a flow diagram illustrating an example method for training a neural network (e.g., an ML model implemented by the neural network) to perform one or more of the tasks as described with respect to some embodiments provided herein.

FIG. 7 is a simplified block diagram illustrating an example system or apparatus for performing one or more of the tasks as described with respect to some embodiments provided herein.

### DETAILED DESCRIPTION

The present disclosure is illustrated by way of example, and not by way of limitation, in the figures of the accompanying drawings.

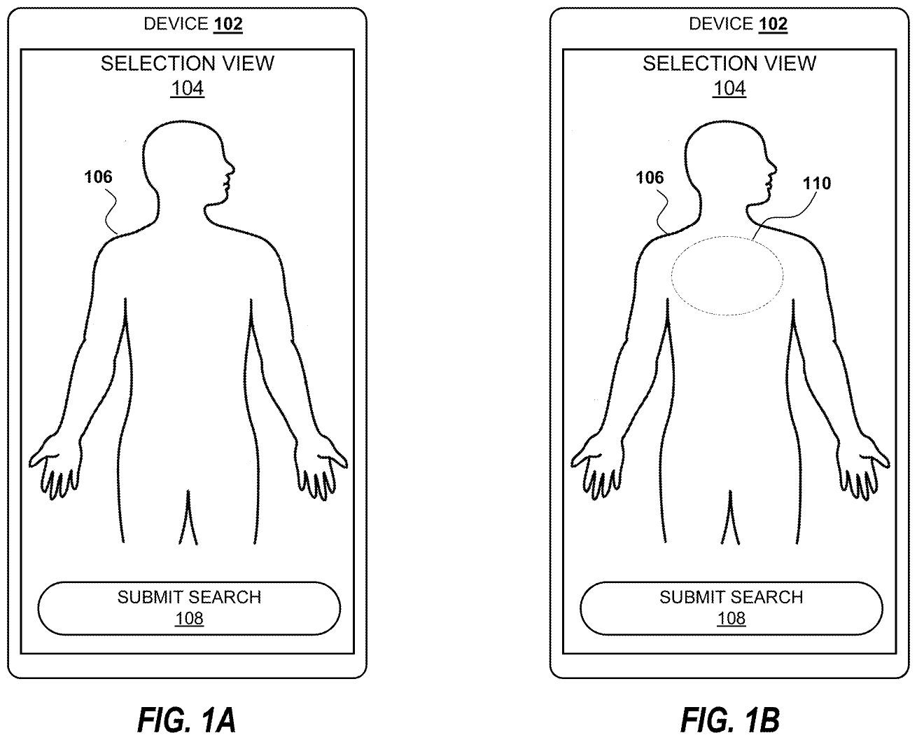

FIGS. 1A-1B show simplified diagrams illustrating examples of graphical user interfaces (GUIs) for accessing and visually interacting with a patient's medical records, according to embodiments described herein.

As shown in FIG. 1A, a device 102 (e.g., a mobile device such as a smartphone) may be configured to display an interface 104, for example, as part of a GUI for a medical records application (e.g., a medical records smartphone app or a web portal) that may allow medical facilities to securely share a patient's medical records, scan images, and/or related data (e.g., through secure login information unique to each patient). Embodiments may be described herein using a mobile app as an example, but those skilled in the art will appreciate that the same or similar techniques may also be employed by other systems, apparatus, or instrumentalities including, e.g., a web server and/or a desktop or laptop computer. Those skilled in the art will also appreciate that, in addition to interacting with an application interface (API) provided by a medical facility, the medical records application may also collect and store data from other wellness/health APIs (e.g., personal health/wellness sensors, results from genetic testing, etc.). In examples, the medical records application may store the heterogeneous medical/health/wellness records of each patient, from the variety of sources (providers, records, manual inputs, etc.), onto the patient's personal device(s) such as the device 102.

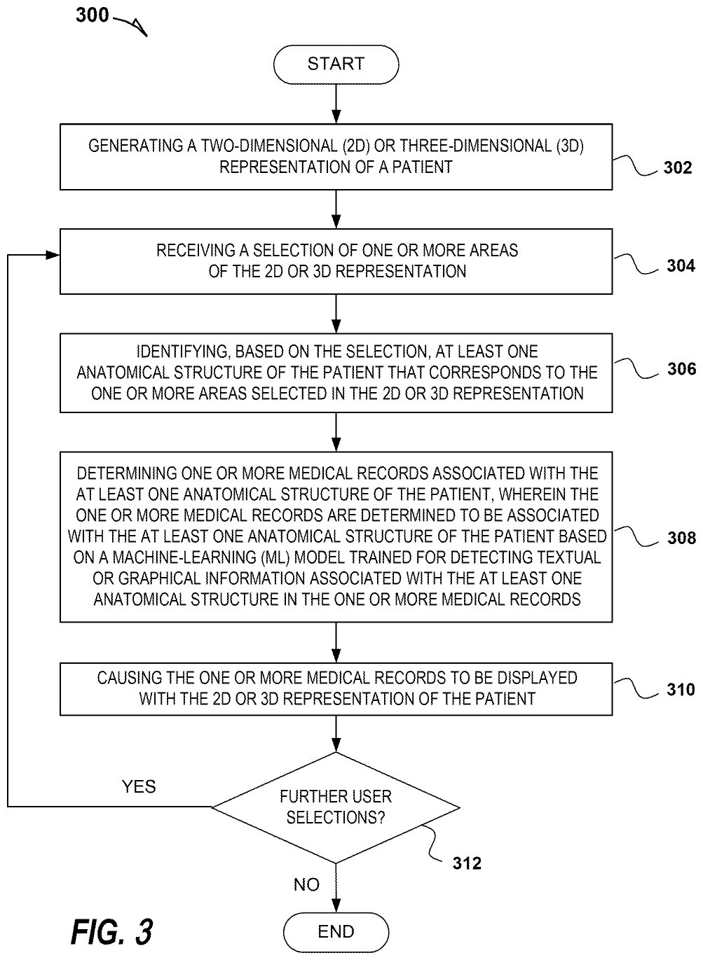

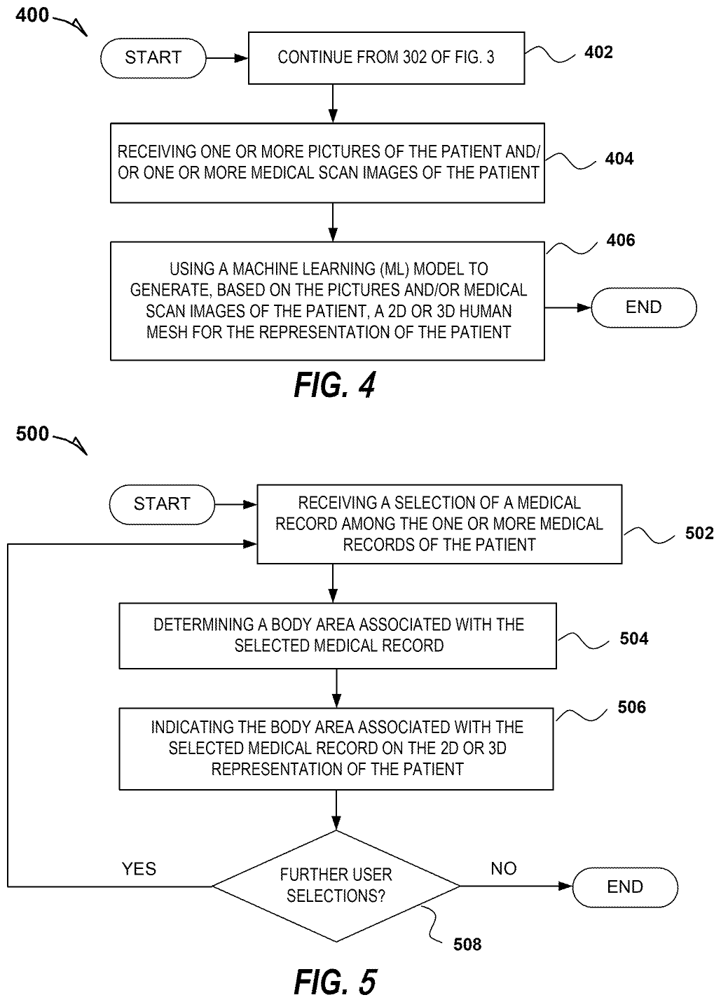

As shown in FIG. 1A, interface 104 may include an interactive graphical representation 106 of a patient's body (e.g., a 2D or 3D representation) configured to receive a selection from a user of the medical records application such as a patient or a doctor. This interactive graphical representation 106 may be a generic human model (e.g., a generic 2D or 3D mesh) or it may be a model specific to the patient, e.g., generated based on one or more images of the patient captured by a camera installed in a medical environment and used upon the patient entering the medical environment as explained more fully below with respect to the system of FIG. 3. In some embodiments, the medical records application may customize and/or refine the interactive graphical representation 106 of the patient's body from collected data, e.g., leveraging collected body metrics (e.g., height, weight, fat percentage, biological sex, etc.) and/or images (e.g., medical scans) to personalize the appearance of the graphical representation 106. In this regard, the medical records application may use a machine-learning (ML) model (e.g., which may refer to the structure and/or parameters of an artificial neural network (ANN) trained to perform a task) to take as input some of the patient's health/body metrics (e.g., height, weight, etc.), personal information (e.g., age, biological sex, etc.), medical scans, and/or color images of the patient (e.g., “selfies”/portraits, full-body pictures, etc.), to create a personalized 3D representation of the patient's body and/or anatomy as the interactive graphical representation 106. In some embodiments, any information that may not be available to the ML model for building a realistic 2D or 3D representation of the patient's appearance and anatomy for the interactive graphical representation 106 (e.g., if the user's age is unknown), may be substituted with pre-defined default parameters to build an approximated representation of the patient.

In some embodiments, the medical records application may allow the user to interact with the GUI in order to visualize the graphical representation 106 from different viewpoints using controls for rotation, zoom, translation, etc. The user may also select (e.g., switch) between different views of the graphical representation 106 based on different layers of the representation such as displaying the body surface (e.g., the external color appearance) of the patient or displaying different anatomical structures (e.g., organs, muscles, skeleton, etc.) of the patient.

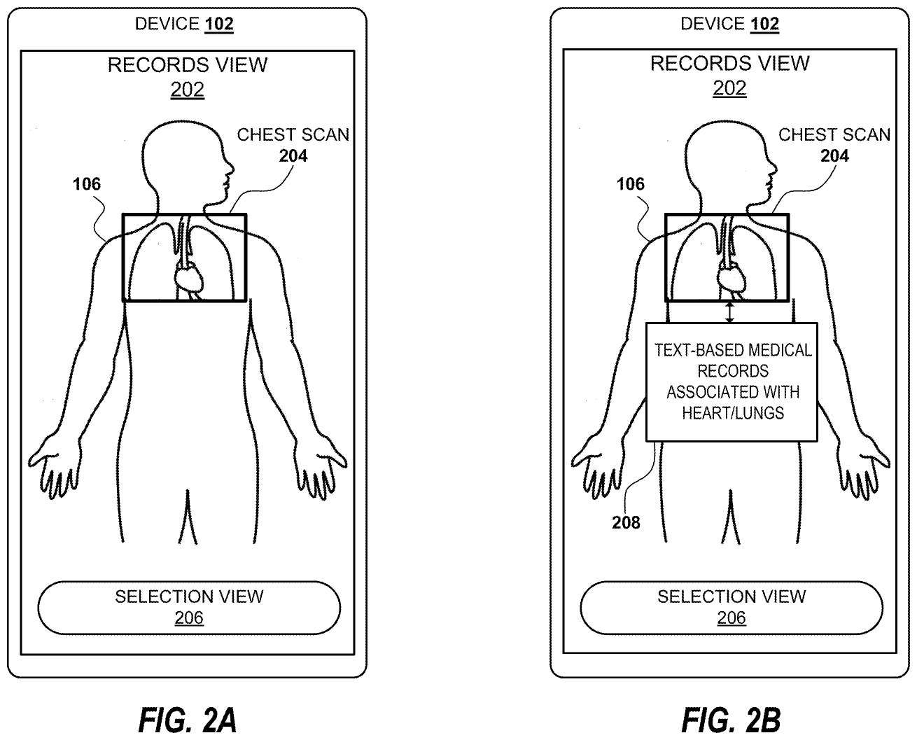

In some embodiments, the selection view interface 104 may include a “submit search” button 108 that may be pressed by the user (e.g., the patient) to query their medical records after having selected one or more specific body areas (e.g., head and/or chest) on the graphical representation 106, or vice-versa highlighting a specific body area relating to a selected medical record (e.g., via a medical record selection interface of the medical records application GUI that is not shown). Still further medical image scans and/or their annotations may be mapped to the selected areas and displayed together with the interactive graphical representation 106 of the patient's body, as described more fully below with respect to FIGS. 2A-2B. In some embodiments, the selection view interface 104 may not include the “submit search” button and the query for the patient's medical records may be submitted upon the user selecting (e.g., clicking, circling, etc.) one or more specific body areas on the graphical representation 106.

As shown in FIG. 1B, the device 102 (e.g., a smartphone) may be configured to display the selection view interface 104 as part of the GUI for the medical records application including the interactive graphical representation 106 to receive at least one selection 110 from a user of the medical records application. The user selection 110 may comprise the user clicking on the graphical representation 106 to select a specified area (within which the click occurred) of the patient's body, or the user circling the area on the interactive graphical representation 106. As noted above, the “submit search” button 108 may be pressed by the user (e.g., the patient) to query their medical records for records that are associated with the at least one specific body areas (e.g., head or chest) associated with the user selection 110 (e.g., an encircled area) on the graphical representation 106, or the query may be submitted in response to the user selection 110 without providing or requiring the user to press the “submit search” button.

FIGS. 2A-2B show simplified diagrams further illustrating the examples of GUIs for interacting with the patient's medical records, according to some embodiments described herein.

As shown in FIG. 2A, the device 102 (e.g., a smartphone) may display an interface 102 (e.g., a medical “records view” interface) as part of the GUI for the medical records application (e.g., a medical records web application viewed in a web browser). The records view interface 202 may include the interactive graphical representation 106 displayed together with an image of at least one medical record (e.g., medical scan image of chest 204) associated with an anatomical structure (e.g., heart or lungs) that is located within (or otherwise associated with) the area of interactive graphical representation 106 selected based on user selection 110 of FIG. 1B described above. In some embodiments, this may involve automatically determining what organs lie within (or are otherwise associated with) the selected areas of the interactive graphical representation 106. Any patient data from medical records in numerous forms, from x-ray and magnetic resonance images to blood test concentrations and electrocardiograph data (and their annotations) may be displayed with (e.g., overlaying) the interactive graphical representation 106. For example, a medical scan image (e.g., chest scan 204) may be displayed over the selected area (e.g., chest) of interactive graphical representation 106 so that the anatomical structure associated with the medical record (e.g., heart or lungs) is displayed over the location of interactive graphical representation 106 that would show that anatomical structure in the GUI of the medical application.

The medical records application may analyze the medical records to determine whether they are related to one or more anatomical structures in the selected area(s). This analysis may be performed based on one or more machine-learning (ML) models (e.g., an artificial neural network(s) used to learn and implement the one or more ML models) including, e.g., a natural language processing model, an image classification model, an image segmentation model, etc. For instance, the natural language processing model may be trained to automatically recognize that a medical record is related to an anatomical structure in the selected area(s) based on texts contained in the medical record. For example, the natural language processing model may link medical records containing the word “migraine” to the “head” area of a patient. In this way textual medical records (e.g., diagnoses, narratives, prescriptions, etc.) may be parsed using the model to process text to identify the organs/body parts that these medical records are associated with (e.g., linking a diagnosis referring to “coughing” to the “lungs” region, linking a “heart rate” metric to the “heart” or the “chest” area of the patient, linking “glucose level” to the “liver” or the “midsection” area of the patient, etc.). As another example, an image classification and/or segmentation model may be trained to process medical scan images to identify the anatomical regions (e.g., head or chest) and/or the anatomical structures (e.g., heart or lungs) that may appear in the medical scan images, e.g., recognizing that a CT scan of the patient may be for the “head” area and/or the “brain” of the patient. In examples, if multiple scan images (e.g., from different image modalities) related to the selected area(s) are identified, these scan images may be registered (e.g., via translation, rotation, and/or scaling) so that they may be aligned with each other and/or with the selected area(s) before being displayed in the interactive graphical representation 106 (e.g., overlaid with the interactive graphical representation 106).

In examples, the records view interface 202 may include a “selection view” button 206 that may be pressed by the user (e.g., the patient) to return to the selection view interface 104, described above with respect to FIGS. 1A-1B, in order to further query their medical records (e.g., via the search button 108) after having selected (e.g., user selection 110) at least one specific body area (e.g., head or chest) on the graphical representation 106.

As shown in FIG. 2B, in some embodiments, the device 102 may display the medical “records view” interface 202 as part of the GUI for the medical records application including the interactive graphical representation 106 displayed together with an overlaid image of one or more medical records (e.g., a medical scan image of chest 204) as described above with respect to FIG. 1. In some embodiments, this may involve also displaying (e.g., overlaying) text-based medical records with the interactive graphical representation 106. For example, a medical scan image (e.g., chest scan 204) may be displayed over the selected area (e.g., chest) of interactive graphical representation 106 and one or more text based medical records 208 that are related to the anatomical structure (e.g., heart or lungs) associated with the image-based medical record (e.g., medical scan image of chest 204) may be displayed nearby in the GUI of the medical records application so that it may visually indicate that the image-based records and text based records 208 are related to each other. Alternatively, text based medical records 208 that may be related to the anatomical structure (e.g., heart or lungs) associated with the selected area (e.g., head or chest) of interactive graphical representation 106 may be shown over the selected area without any associated image based medical records.

As described herein, the interactive graphical representation 106 shown in FIGS. 1A-2B may include a 2D or 3D human model of the patient that may be created using an artificial neural network (ANN). In examples, the ANN may be trained to predict the 2D or 3D human model based on images (e.g., 2D images) of the patient that may be stored by a medical facility or uploaded by the patient. For instance, given an input image (e.g., a color image) of the patient, the ANN may extract a plurality of features, Φ, from the image and provide the extracted features to a human pose/shape regression module configured to infer parameters from the extracted features for recovering the 2D or 3D human model. These inferred parameters may include, e.g., pose parameters, Θ, and shape parameters, β, that may respectively indicate the pose and shape of the patient's body. In examples, the pose parameters Θ may include 72 parameters derived based on joint locations of the patient (e.g., 3 parameters for each of 23 joints comprised in a skeletal rig plus three parameters for a root joint), with each parameter corresponding to an axis-angle rotation from a root orientation. The shape parameters β may be learned based on a principal component analysis (PCA) and may include a plurality of coefficients (e.g., the first 10 coefficients) of the PCA space. Once the pose and shape parameters are determined, a plurality of vertices (e.g., 6890 vertices based on 82 shape and pose parameters) may be obtained for constructing a representation (e.g., a 3D mesh) of the human body. Each of the vertices may include respective position, normal, texture, and/or shading information. Using these vertices, a 3D mesh of the person may be created, for example, by connecting multiple vertices with edges to form a polygon (e.g., such as a triangle), connecting multiple polygons to form a surface, using multiple surfaces to determine a 3D shape, and applying texture and/or shading to the surfaces and/or shapes.

In examples, the interactive graphical representation 106 shown in FIGS. 1A-2B may include a view (e.g., provided by a layer of the graphical representation) of an anatomical structure (e.g., an organ) of the patient in addition to a view of the body surface (e.g., external appearance) of the patient. Such a view or layer of the anatomical structure may be created using an artificial neural network (e.g., based on an ML model learned by the neural network) training for automatically predicting the geometrical characteristics (e.g., a contour) of the anatomical structure based on the physical characteristics (e.g., body shape and/or pose) of the patient. In examples, the artificial neural network may be trained to perform this task based on medical scan images of the anatomical structure and a statistical shape model of the anatomical structure. The statistical shape model may include a mean shape of the anatomical structure (e.g., a mean point cloud indicating the shape of the anatomical structure) and a principal component matrix that may be used to determine the shape of the anatomical structure depicted by the one or more scan images (e.g., as a variation of the mean shape). The statistical shape model may be predetermined, for example, based on sample scan images of the anatomical structure collected from a certain population or cohort and segmentation masks of the anatomical structure corresponding to the sample scan images. The segmentation masks may be registered with each other via affine transformations and the registered segmentation masks may be averaged to determine a mean point cloud representing the mean shape of the anatomical structure. Based on the mean point cloud, a respective point cloud may be derived in the image domain for each sample scan image, for example, through inverse deformation and/or transformation. The derived point clouds may then be used to determine a principal component matrix, for example, by extracting the principal modes of variations to the mean shape. In examples, the artificial neural network may be trained to determine the correlation (e.g., a spatial relationship) between the geometric characteristics (e.g., shape and/or location) of the anatomical structure and the body shape and/or the pose of the patient, and represent the correlation through a plurality of parameters that may indicate how the geometric characteristics of the anatomical structure may change in accordance with changes in the patient's body shape and/or pose. Examples of such an artificial neural network can be found in commonly assigned U.S. patent application Ser. No. 17/538,232, filed Nov. 30, 2021, entitled “Automatic Organ Geometry Determination,” the disclosure of which is hereby incorporated by reference in its entirety.

The image classification, object segmentation, and/or natural language processing tasks described herein may also be accomplished using one or more ML models (e.g., using respective ANNs that implement the ML models). For example, the medical records application described herein may be configured to determine that one or more medical scan images may be associated with an anatomical structure of the patient using an image classification and/or segmentation neural network trained for detecting the presence of the anatomical structure in the medical scan images. The training of such a neural network may involve providing a set of training images of anatomical structures (e.g., referred to herein as a training set) and force the neural network to learn from the training set what every one of the anatomical structures looks like and/or where the contour of each anatomical structure is such that when given an input image, the neural network may predict which one or more the anatomical structures are contained in the input image (e.g., by generating a label or segmentation mask for the input image). The parameters of the neural network (e.g., corresponding to an ML model as described herein) may be adjusted during the training by comparing the true labels or segmentation masks of these training images (e.g., which may be referred to as the ground truth) to the ones predicted by the neural network.

As another example, the medical records application may also be configured to determine that one or more text-based medical records may be associated with an anatomical structure of the patient using a natural language processing (NPL) neural network trained for linking certain texts in the medical records with the anatomical structure (e.g., based on textual information extracted by the neural network from the medical records). In some example implementations, the NPL neural network may be trained to classify (e.g., label) the texts contained in the medical records as belonging to respective categories (e.g., a set of anatomical structures of the human body, which may be predefined). Such a network may be trained, for example, in a supervised manner, based on training datasets that may include pairs of input text and ground-truth label. In other example implementations, the NPL neural network may be trained to extract structured information from the medical records and answer more broadly predefined questions such as what anatomical structure(s) the text in the medical records refers to.

The artificial neural network described herein may include a convolutional neural network (CNN), a multilayer perceptron (MLP) neural network, and/or another suitable type of neural networks. The artificial neural network may include multiple layers such as an input layer, one or more convolutional layers, one or more pooling layers, one or more fully connected layers, and/or an output layer. Each of the layers may include a plurality of filters (e.g., kernels) having respective weights configured to detect (e.g., extract) a respective feature or pattern from the input image (e.g., the filters may be configured to produce an output indicating whether the feature or pattern has been detected). The weights of the neural network may be learned by processing a training dataset (e.g., comprising images or texts) through a training process that will be described in greater detail below.

FIG. 3 shows a flow diagram illustrating an example method 300 for determining medical records of a patient based on selected areas of a graphical representation (e.g., 2D or 3D) of the patient and displaying the records together with the graphical representation, according to embodiments described herein.

As show, the method 300 may generate a 2D or 3D representation of the patient (e.g., interactive graphical representation 106 of FIG. 1A for the GUI of a medical records application) at 302. As noted above, the 2D or 3D representation may include a 3D human mesh generated using an ML model trained for recovering a 2D or 3D human mesh based on one or more pictures of the patient or one or more medical scan images of the patient. Furthermore, the 2D or 3D human mesh of the patient may be modified based on the one or more medical records selected from a medical record repository (e.g., using an image classification/segmentation ML model and/or a natural language processing ML model described with respect to operation 308 below). In some embodiments, the 2D or 3D representation of the patient includes a first view depicting a body surface of the patient and a second view depicting one or more anatomical structures of the patient, and a user may switch from displaying the first view of the patient to displaying the second view of the patient.

At 304, a selection of one or more areas of the 2D or 3D representation of the patient (e.g., interactive graphical representation 106) may be received (e.g., by the medical records application). As explained above, the area selection (e.g., user selection 110 of FIG. 1B) may be in the form of clicking on the 2D or 3D representation of the patient or by circling an area of the 2D or 3D representation of the patient (e.g., with a mouse, a finger or an electronic pen/pencil). Specified areas (e.g., head or chest) may then be selected based on the user selection and medical records associated with anatomical structure located in this area may be queried (e.g., using the submit search 108 button of FIG. 1A).

At 306, based on the selection (e.g., user selection 110 of FIG. 1B) having been made, at least one anatomical structure (e.g., brain or heart) of the patient that corresponds to the one or more areas of the 2D or 3D representation may be identified. As explained above, this may involve determining what anatomical structures lie within (or are otherwise associated with) the selected areas of the 2D or 3D representation (e.g., graphical representation 106), for example, based on a medical information database that is part of (or accessible to) the medical records application.

At 308, one or more medical records (e.g., chest scan 204 of FIG. 2A) associated with the at least one anatomical structure of the patient (e.g., heart or lungs) may be determined, for example, based on one or more ML models trained for detecting textual or graphical information associated with the at least one anatomical structure in the one or more medical records. In some embodiments, the one or more ML models may include an image classification model trained for automatically recognizing that the one or more medical records include a medical scan image (e.g., chest scan 204 of FIG. 2A) of the at least one anatomical structure (e.g., heart or lungs). In some embodiments, the one or more ML models may include an object segmentation model trained for segmenting the at least one anatomical structure (e.g., heart or lungs) from the medical scan image. The segmentation may allow for the location and/or boundaries of the anatomical structure to be more easily visualized in the records view 202 of FIG. 2A for the GUI of the medical records application. In some embodiments, the one or more ML models may include a text processing model trained for automatically recognizing that the one or more medical records (e.g., text based record 208 for FIG. 2B) include terms associated with the at least one anatomical structure (e.g., “heart” or “lungs”).

At 310, the one or more medical records (e.g., chest scan 205 of FIG. 1A and/or text based medical records 208) may be presented (e.g., displayed), for example, with the 2D or 3D representation of the patient (e.g., interactive graphical representation 106 of FIG. 1A). As noted above, displaying the one or more medical records with the 2D or 3D representation of the patient may comprise overlaying the 2D or 3D representation of the patient with the one or more medical records and displaying the 2D or 3D representation of the patient overlaid with the one or more medical records. In some embodiments, displaying the one or more medical records with the 2D or 3D representation of the patient comprises registering respective medical scan images associated with multiple anatomical structures and displaying the registered medical scan images with the 2D or 3D representation of the patient. At 312, a determination may be made regarding whether further user selections are received. If the determination is that a further selection is received, the method 300 may return to 304; otherwise, the method 300 may end.

FIG. 4 shows a flow diagram illustrating an example method 400 for generating the 2D or 3D human mesh to represent the patient based on pictures and/or medical scan image(s) of the patient, according to some of the embodiments described herein.

As shown, the method 400 may start at 402, for example, as part of operation 302 illustrated by FIG. 3, and may include obtaining, at 404, one or more pictures (e.g., color pictures) of the patient and/or one or more medical scan images of the patient (e.g., the scan images may include magnetic resonance imaging (MRI) images and/or computed tomography (CT) images of the patient). The pictures and/or images of the patient may be captured during the patient's previous visits to a medical facility, or they may be uploaded by the patient or a doctor to the medical records application described herein. Based on the pictures and/or medical scan images of the patient, an ML model may be used at 406 to generate a 2D or 3D human mesh as a representation of the patient (e.g., interactive graphical representation 106 of FIG. 1A). As explained above, such an ML model may be trained to take as input pictures and/or medical scan images of the patient, analyze the pictures and/or images to determine parameters that may indicate the pose and/or shape of the patient, and create a personalized representation of the patient's body and anatomy as the interactive graphical representation 106.

FIG. 5 shows a flow diagram illustrating an example method 500 for determining areas of the graphical representation of the patient corresponding to a selected medical record of the patient and indicating the determined areas on the graphical representation, according to some embodiments described herein.

The method 500 may include receiving, at 502, a selection of a medical record from among one or more medical records of the patient (e.g., via a medical records selection interface of the GUI of the medical records application), and determining, at 504, a body area (e.g., head or chest) that may be associated with the selected medical record. This may involve determining, using one or more ML models such as the image classification/segmentation model or the text processing model described herein, what anatomical structures are associated with the selected medical record, and further determining what body areas of the 2D or 3D representation (e.g., graphical representation 106) the associated anatomical structures lie within (or are otherwise associated with). The latter determination may be made, for example, based on a mapping relationship between areas of a human body and anatomical structures of the human body. Once determined, the body area(s) associated with the selected medical record may be indicated (e.g., highlighted or otherwise distinguished) on the 2D or 3D representation of the patient at 506. As explained above with respect to FIGS. 1A-1B, the user may then click on (or otherwise select) the indicated area of the 2D or 3D representation to search for (e.g., submit search button 108 of FIG. 1A) other medical records that may be associated with the indicated area. Then, at 508, a determination may be made regarding whether further user selections are received. If the determination is that a further user selection is received, the method 500 may return to 504; otherwise, the method 500 may end.

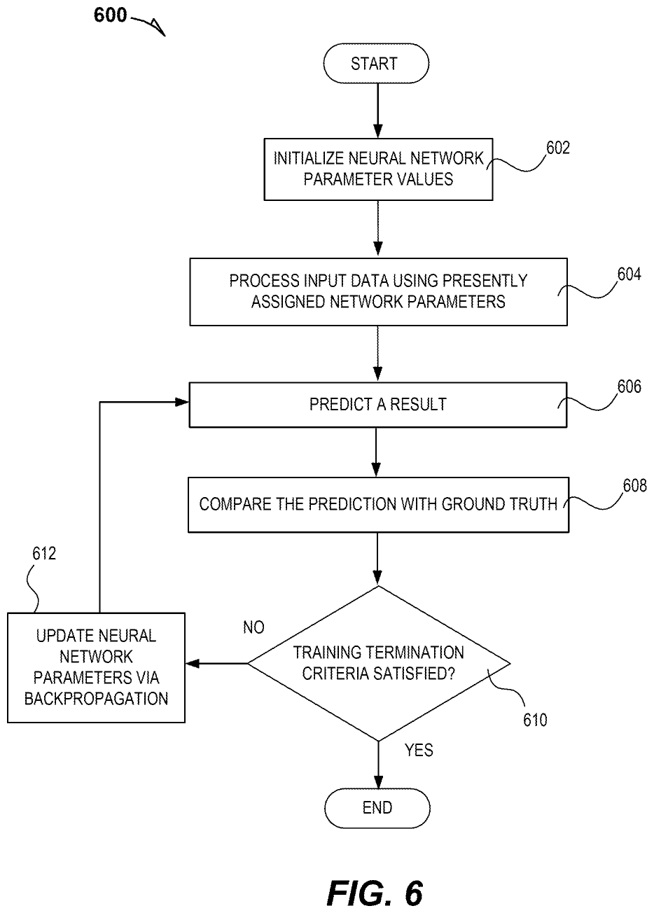

FIG. 6 shows a flow diagram illustrating an example method 600 for training a neural network (e.g., an ML model implemented by the neural network) to perform one or more of the tasks described herein. As shown, the training method 600 may include, at 602, initializing the operating parameters of the neural network (e.g., weights associated with various layers of the neural network), for example, by sampling from a probability distribution or by copying the parameters of another neural network having a similar structure. The training method 600 may further include processing an input (e.g., a training image) using presently assigned parameters of the neural network at 604, and making a prediction for a desired result (e.g., an image classification or segmentation, a text processing result, etc.) at 606. The prediction result may be compared to a ground truth at 608 to determine a loss associated with the prediction, for example, based on a loss function such as mean squared errors between the prediction result and the ground truth, an L1 norm, an L2 norm, etc. At 610, the loss may be used to determine whether one or more training termination criteria are satisfied. For example, the training termination criteria may be determined to be satisfied if the loss is below a threshold value or if the change in the loss between two training iterations falls below a threshold value. If the determination at 610 is that the termination criteria are satisfied, the training may end; otherwise, the presently assigned network parameters may be adjusted at 612, for example, by backpropagating a gradient descent of the loss function through the network before the training returns to 606.

For simplicity of explanation, the training steps are depicted and described herein with a specific order. It should be appreciated, however, that the training operations may occur in various orders, concurrently, and/or with other operations not presented or described herein. Furthermore, it should be noted that not all operations that may be included in the training method are depicted and described herein, and not all illustrated operations are required to be performed

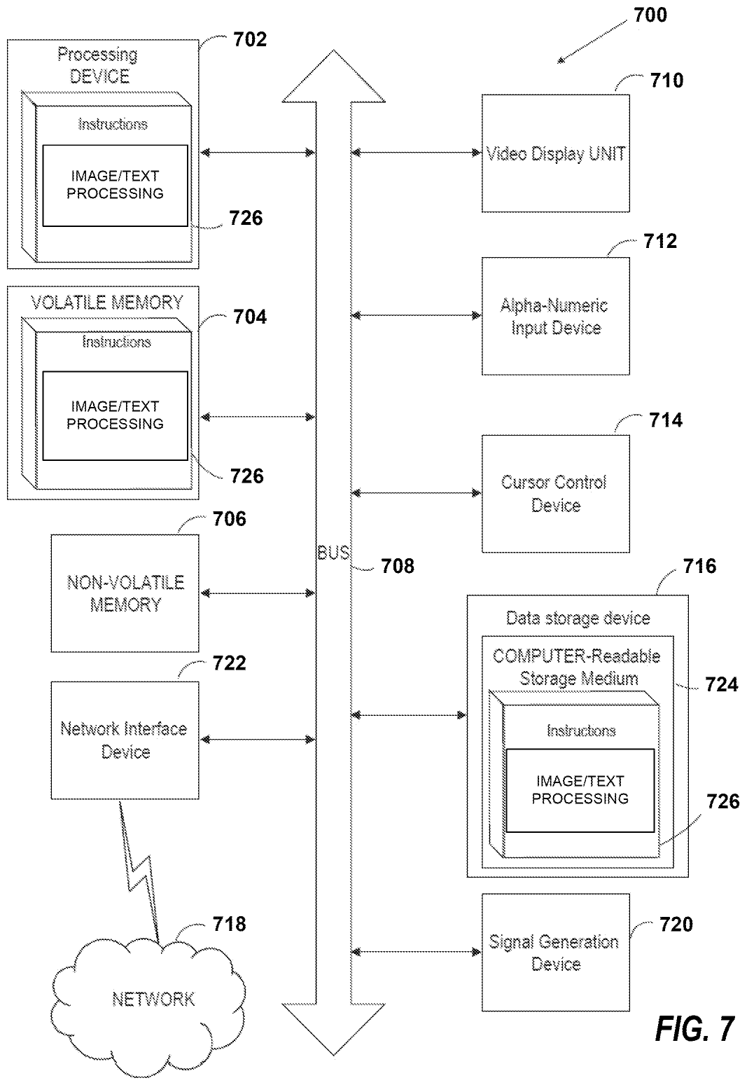

FIG. 7 shows a simplified block diagram illustrating an example system or apparatus 700 for performing one or more of the tasks described herein. In embodiments, apparatus 700 may be connected (e.g., via a network 718, such as a Local Area Network (LAN), an intranet, an extranet, or the Internet) to other computer systems. Apparatus 700 may operate in the capacity of a server or a client computer in a client-server environment, or as a peer computer in a peer-to-peer or distributed network environment. Apparatus 700 may be provided by a personal computer (PC), a tablet PC, a set-top box (STB), a Personal Digital Assistant (PDA), a cellular telephone, a web appliance, a server, a network router, switch or bridge, or any device capable of executing a set of instructions (sequential or otherwise) that specify actions to be taken by that device (e.g., device 102 of FIG. 1). Further, the term “computer” shall include any collection of computers that individually or jointly execute a set (or multiple sets) of instructions to perform any one or more of the methods described herein.

Furthermore, apparatus 700 may include a processing device 702 (e.g., one or more processors), a volatile memory 704 (e.g., random access memory (RAM)), a non-volatile memory 706 (e.g., read-only memory (ROM) or electrically-erasable programmable ROM (EEPROM)), and/or a data storage device 716, which may communicate with each other via a bus 708. Processing device 702 may include one or more processors such as a general purpose processor (e.g., such as a complex instruction set computing (CISC) microprocessor, a reduced instruction set computing (RISC) microprocessor, a very long instruction word (VLIW) microprocessor, a microprocessor implementing other types of instruction sets, or a microprocessor implementing a combination of types of instruction sets) or a specialized processor (e.g., such as an application specific integrated circuit (ASIC), a field programmable gate array (FPGA), a digital signal processor (DSP), or a network processor).

Apparatus 700 may further include a network interface device 722, a video display unit 710 (e.g., an LCD), an alphanumeric input device 712 (e.g., a keyboard), a cursor control device 714 (e.g., a mouse), a data storage device 716, and/or a signal generation device 720. Data storage device 716 may include a non-transitory computer-readable storage medium 724 on which instructions 726 encoding any one or more of the image/text processing methods or functions described herein may be stored. Instructions 726 may also reside, completely or partially, within volatile memory 704 and/or within processing device 702 during execution thereof by apparatus 700, hence, volatile memory 704 and processing device 702 may comprise machine-readable storage media.

While computer-readable storage medium 724 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description.

What is claimed is:

1. An apparatus, comprising:
one or more processors, wherein the one or more processors are configured to:
generate a two-dimensional (2D) or three-dimensional (3D) representation of a patient;
receive a selection of one or more areas of the 2D or 3D representation;
identify, based on the selection, at least one anatomical structure of the patient that corresponds to the one or more areas of the 2D or 3D representation;
determine one or more medical records associated with the at least one anatomical structure of the patient, wherein the one or more medical records are determined to be associated with the at least one anatomical structure of the patient using a first machine-learning (ML) model trained for detecting textual or graphical information associated with the at least one anatomical structure in the one or more medical records; and
present the one or more medical records.

2. The apparatus of claim 1, wherein the 2D or 3D representation includes a 2D or 3D human mesh, and wherein the one or more processors are configured to generate the 2D or 3D human mesh using a second ML model trained for recovering the 2D or 3D human mesh based on one or more pictures of the patient or one or more medical scan images of the patient.

3. The apparatus of claim 2, wherein the one or more processors are further configured to modify the 2D or 3D human mesh of the patient based on the one or more medical records determined by the first ML model.

4. The apparatus of claim 1, wherein the one or more processors being configured to present the one or more medical records comprises the one or more processors being configured to overlay the 2D or 3D representation of the patient with the one or more medical records and display the 2D or 3D representation of the patient overlaid with the one or more medical records.

5. The apparatus of claim 1, wherein the one or more medical records comprise medical scan images of the patients, and wherein one or more processors being configured to present the one or more medical records comprises the one or more processors being configured to register the medical scan images and display the registered medical scan images together with the 2D or 3D representation of the patient.

6. The apparatus of claim 1, wherein the one or more medical records include a medical scan image of patient, and the first ML model includes an image classification model trained for automatically recognizing that the medical scan image is associated with the at least one anatomical structure of the patient.

7. The apparatus of claim 6, wherein the first ML model is further trained to segment the at least one anatomical structure from the medical scan image.

8. The apparatus of claim 1, wherein the one or more medical records include a diagnosis or prescription for the patient, and the first ML model includes a text processing model trained for automatically recognizing that the diagnosis or prescription includes texts associated with the at least one anatomical structure of the patient.

9. The apparatus of claim 1, wherein the 2D or 3D representation of the patient includes multiple views of patient and wherein the one or more processors are further configured to switch from presenting a first view of the patient to presenting a second view of the patient based on a user input.

10. The apparatus of claim 9, wherein the first view depicts a body surface of the patient and the second view depicts one or more anatomical structures of the patient.

11. The apparatus of claim 1, wherein the one or more processors are further configured to:
receive a selection of a medical record among the one or more medical records of the patient;
determine a body area of the patient associated with the selected medical record;
indicate the body area associated with the selected medical record on the 2D or 3D representation of the patient.

12. A method for presenting medical information, the method comprising:
generating a two-dimensional (2D) or three-dimensional (3D) representation of a patient;
receiving a selection of one or more areas of the 2D or 3D representation;
identifying, based on the selection, at least one anatomical structure of the patient that corresponds to the one or more areas of the 2D or 3D representation;
determining one or more medical records associated with the at least one anatomical structure of the patient, wherein the one or more medical records are determined to be associated with the at least one anatomical structure of the patient using a first machine-learning (ML) model trained for detecting textual or graphical information associated with the at least one anatomical structure in the one or more medical records; and
presenting the one or more medical records.

13. The method of claim 12, wherein the 2D or 3D representation includes a 2D or 3D human mesh, and wherein the 2D or 3D human mesh is generated using a second ML model trained for recovering the 2D or 3D human mesh based on one or more pictures of the patient or one or more medical scan images of the patient.

14. The method of claim 12, further comprising modifying the 2D or 3D human mesh of the patient based on the one or more medical records determined by the first ML model.

15. The method of claim 12, wherein presenting the one or more medical records comprises overlaying the 2D or 3D representation of the patient with the one or more medical records and displaying the 2D or 3D representation of the patient overlaid with the one or more medical records.

16. The method of claim 12, wherein the one or more medical records include a medical scan image of patient, and the first ML model includes an image classification model trained for automatically recognizing that the medical scan image is associated with the at least one anatomical structure of the patient.

17. The method of claim 17, wherein the first ML model is further trained to segment the at least one anatomical structure from the medical scan image.

18. The method of claim 12, wherein the one or more medical records include a diagnosis or a prescription for the patient, and the first ML model includes a text processing model trained for automatically recognizing that the diagnosis or prescription includes texts associated with the at least one anatomical structure of the patient.

19. The method of claim 12, wherein the 2D or 3D representation of the patient includes multiple views of patient, and wherein the method further comprises switching from presenting a first view of the patient to presenting a second view of the patient based on a user input, the first view depicting a body surface of the patient, the second view depicting one or more anatomical structures of the patient.

20. The method of claim 12, further comprising:
receiving a selection of a medical record among the one or more medical records of the patient;
determining a body area of the patient associated with the selected medical record;
indicating the body area associated with the selected medical record on the 2D or 3D representation of the patient.
